(19) United States
(12) Patent Application Publication
(10) Pub. No.: US 2021/0247394 A1
(43) Pub. Date: Aug. 12, 2021

(54) ISOLATED BACTERIAL STRAIN FOR INDUCING PROLIFERATION OR ACCUMULATION OF REGULATORY T-CELLS

(71) Applicant: ICAHN SCHOOL OF MEDICINE AT MOUNT SINAI, New York, NY (US)

(72) Inventors: Graham BRITTON, New York, NY (US); Jeremiah FAITH, New York, NY (US); Zhihua LI, Summit, NJ (US); Ilaria MOGNO, New York, NY (US)

(73) Assignee: Icahn School of Medicine at Mount Sinai, New York, NY (US)

(21) Appl. No.: 17/049,012

(22) PCT Filed: Apr. 19, 2019
(86) PCT No.: PCT/US2019/028238

§ 371 Date: Oct. 19, 2020

Related U.S. Application Data
(60) Provisional application No. 62/662,453, filed on Apr. 25, 2018.

(51) Int. Cl. G01N 33/569; C12Q 1/689; A61K 35/744

(52) U.S. Cl. CPC G01N 33/56916; C12Q 1/689; A61K 2035/115; C12Q 2600/136; A61K 35/744

(57) ABSTRACT

The present invention relates to methods of using bacterial strain 1687A6 in detecting, diagnosing and treating disease or disorders of the GI tract. The present invention also relates to modulating the immune responses of an individual by inducing Th17 cell differentiation, proliferation, or accumulation. Further, the invention relates to therapeutic compositions containing strain 1687A6 or compounds derived from it and methods for treating disease in a subject using such compositions.

### CROSS REFERENCE TO PRIOR APPLICATIONS

This application is a § 371 national stage of PCT International Application No. PCT/US2019/028238, filed Apr. 19, 2019, claiming the benefit of U.S. Provisional Application No. 62/662,453, filed Apr. 25, 2018, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 28, 2019, is named MS-0004-WO-PCT_SL.txt and is 6,387,952 bytes in size.

### FIELD OF THE INVENTION

The present invention relates generally to diagnosing, treating, and monitoring the progression of inflammatory bowel disease in a subject, including, for example, by monitoring treatment. The present invention also relates to a method for inducing or inhibiting proliferation or accumulation of Th17 cells or modulating an immune response, inflammation, or a drug response. Such method may include reduction of inflammation caused by Th17 cells in the gastrointestinal tract via a drug, vaccine, probiotic, or other means. Such method may also include induction or modulation of the immune response in the body in response to a specific microbial strain or to a physiologically active substance derived from that strain. Diagnosis and monitoring can be achieved by tracking biomarkers in the subject that are associated with the specific microbial strain identified.

### BACKGROUND

The mammalian gastrointestinal (GI) tract harbors a diverse microbial community that is usually maintained in symbiotic balance. Interactions between microbes within the microbial populations, and between the microbes and the host, affect both the host and the internal microbial community. For example, a majority of the mammalian immune system is directed at the GI tract. This may affect the internal microbial community structure, by eliminating some microbes and enabling the expansion of others. Even the host diet shapes the GI microbial community often by introducing new microbes, benefitting some microbes, or inhibiting others.

A healthy GI microbiome also benefits the host. It can render the host resistant to colonization by a broad spectrum of pathogens, provide immune stimulation, promote maintenance of a healthy gut epithelium, and provide essential nutrients such as vitamin K for absorption. In some individuals, this symbiotic balance is disrupted. This state can lead to disruption of microbial functions and lead to increased susceptibility to pathogens, induction of inflammatory signaling cascades that result in autoimmunity, and disruption of nutrient absorption. Consequently, the GI microbiome is a significant element in the pathogenesis of many diseases and disorders. Some patients become more susceptible to pathogenic infections after the use of broad-spectrum antibiotics that disrupt the normal intestinal microbiota flora. Many of these diseases and disorders, such as inflammatory bowel disease (IBD) are chronic conditions that significantly decrease a patient's quality of life and can be ultimately fatal.

IBD describes a variety of intestinal disorders, including Crohn's disease (CD) and ulcerative colitis (UC), which are all characterized by dysregulated GI immune responses and localized inflammatory signals in affected patients. Recent research has focused on the presence of specific strains of bacteria and the accumulation of Th17 immune cells in the GI as markers of IBD and of the associated inflammatory signals. One study found that adherent and invasive E. coli strains are enriched in the gut of patients with Crohn's disease. Darfeuille-Michaud, A., et al., (2004) Gastroenterology, 127(2): 412. Some studies have correlated specific bacteria with differentation of Th17 cells in mouse models of IBD. Ivanov, II., et al., (2008) Cell Host Microbe, 4(4): 337 and Tan, T.G., et al., (2016) PNAS USA, 113(50): E8141. And a recent study shows that IgA coated E. coli induce inflammation and Th17 accumulation in the gut of Crohn's disease patients. Viladomiu, M., et al., (2017) Sci Transl Med, 9(376). GI related T-cell activation has also been implicated in anti-cancer therapies. Chaput, et al., (2017) Annals of Oncology 28: 1368. One study has also shown that induction of Th17 cells by gut microbiota provide protection from gut pathogens. Ivanov, II, et al., (2009) Cell 139(3): 485.

But to date, no microbial strain has been found that specifically induces accumulation or proliferation of Th17 cells in the human gut or that causes IBD in a human. Thus, there is a need in the art for improved methods for detecting, staging, and treating subjects for the presence of specific bacteria that induce Th17 associated inflammation in a human and that cause IBD. There is also a need for identifying bacterial strains that stimulate Th17 cells and can be used to modulate inflammation and immune responses. The present invention fulfills these needs by identifying for the first time a specific strain of E. coli that induces Th17 cell differentiation and accumulation in a microbial community and that is associated with IBD disease progression.

### SUMMARY OF THE INVENTION

The present invention identifies for the first time a microbial strain isolated from the human gut that causes Th17 associated inflammation in the gut in the context of a complex existing microbiota.

One aspect of the present invention provides a specific microbe that induces T-cell differentiation and inflammation in the gut of patients with IBD.

Another aspect of the present invention provides a biological marker for diagnosing IBD.

Another aspect of the present invention provides a biological marker for staging or tracking IBD.

Another aspect of the present invention provides a biological marker and a method for treating IBD.

Another aspect of the present invention provides a method of screening for compositions and methods to treat or prevent IBD.

Another aspect of the present invention provides a bacterial strain that can modulate an immune reaction in a subject.

Another aspect of the present invention provides a therapeutic composition for modulating an immune response in a subject.

Another aspect of the present invention provides a therapeutic composition for treatment of a subject alone or in combination with other therapeutic compositions to stimulate an immune response.

Another aspect of the present invention provides a method for inducing proliferation or accumulation of Th17 cells, as well as a method for inhibiting such proliferation or accumulation.

Another aspect of the present invention provides a vaccine composition containing a bacterial strain or a physiologically active substance derived from it.

Another aspect of the present invention provides a method for treating or preventing at least one disease or condition selected from infectious diseases, cancer, and autoimmune diseases.

Another aspect of the present invention provides a method for identifying strain specific substances that induce the differentiation, accumulation, or proliferation of Th17 cells.

The invention provides a method in which the absolute amount or the ratio of Escherichia coli 1687A6 (“strain 1687A6”) bacteria in a microbiota of an individual with IBD is determined and can be compared with the baseline value of the amount of strain 1687A6 in a healthy individual to provide an IBD diagnosis, prognosis, or treatment regimen.

In one embodiment, the invention provides a method in which the absolute amount or the ratio of strain 1687A6 bacteria in a microbiota of an individual with IBD is determined, and, when a therapeutically active composition is administered to the individual that reduces the ratio or the absolute value of the strain 1687A6 bacteria in comparison with a base line value in a healthy individual, it is determined that the therapeutically active composition is effective in treating IBD.

In one embodiment, the method further comprises measuring the levels of strain 1687A6 in the microbiota of the subject after administration of a therapeutic composition, wherein an increase in the percentage or absolute number of strain 1687A6 after administration of the therapeutic composition relative to levels prior to the administration is a positive indicator of enhanced immunosuppression (or immunoregulation). The measurement of the composition of the subject's microbiota can be made with techniques known in the art, such as metagenomic sequencing.

One embodiment of the present invention provides a method for detecting strain 1687A6 in a fecal sample by: (a) obtaining a fecal sample from a patient exhibiting symptoms associated with IBD; (b) performing metagenomic sequencing on the sample; (c) detecting whether strain 1687A6 is present in the sample.

One embodiment of the present invention provides a method for detecting strain 1687A6 in a fecal sample by: (a) obtaining a fecal sample from a patient exhibiting symptoms associated with IBD; (b) performing RT-PCR on the sample; (c) detecting the amount of strain 1687A6 present in the sample.

One embodiment of the present invention provides a method for detecting strain 1687A6 in a fecal sample by: (a) obtaining a fecal sample from a patient exhibiting symptoms associated with IBD; (b) contacting the sample with an antibody that binds to strain 1687A6; (c) detecting binding of the antibody with strain 1687A6 in the sample.

One embodiment of the present invention provides a method for diagnosing IBD in a patient by: (a) obtaining a fecal sample from a patient exhibiting symptoms associated with IBD; (b) contacting the sample with an antibody that binds to strain 1687A6; (c) detecting binding of the antibody with strain 1687A6 in the sample; and (d) predicting the presence or assessing status of IBD when the level of strain 1687A6 in the patient is higher than a predetermined level of strain 1687A6.

One embodiment of the present invention provides a method for diagnosing IBD in a patient by: (a) obtaining a fecal sample from a patient exhibiting symptoms associated with IBD; (b) detecting the presence of a strain 1687A6 specific nucleotide in the sample; and (c) predicting the presence or assessing status of IBD when the level of at least one strain 1687A6 specific nucleotide in the patient is higher than a predetermined level of the strain 1687A6 specific nucleotide.

One embodiment of the present invention provides a method of screening for a therapeutic composition useful for treating IBD by: (a) infecting an individual with strain 1687A6; (b) allowing strain 1687A6 to colonize the gut of the individual until the individual exhibits symptoms associated with IBD in that type of individual; (c) treating the individual with a therapeutic composition; and (d) determining whether the therapeutic composition reduces the amount of strain 1687A6 present in the individual below a predetermined level.

One embodiment of the present invention provides a method of screening for a therapeutic composition useful for treating IBD by: (a) infecting an individual with strain 1687A6; (b) allowing strain 1687A6 to colonize the gut of the individual until the individual exhibits symptoms associated with IBD in that type of individual; (c) treating the individual with a therapeutic composition; and (d) comparing the amount of strain 1687A6 present in the infected individual to the amount of strain 1687A6 in a healthy individual.

One embodiment of the present invention provides a method of screening for therapeutic compositions useful for treating IBD by: (a) infecting an individual with strain 1687A6; (b) allowing strain 1687A6 to colonize the gut of the individual; (c) assaying the levels of Th17 cells in the GI of the individual; (c) treating the individual with a therapeutic composition; (d) determining whether the therapeutic composition reduces the levels of Th17 cells present in the GI of the individual below a predetermined level.

One embodiment of the present invention provides a method of screening for a therapeutic composition useful for treating IBD by: (a) infecting an individual with strain 1687A6; (b) allowing strain 1687A6 to colonize the gut of the individual until the individual exhibits symptoms associated with IBD in that type of individual; (c) assaying the levels of Th17 cells in the GI of the individual; (d) treating the individual with a therapeutic composition; and (e) comparing the amount of Th17 cells present in the infected individual to the amount of Th17 cells in a healthy individual.

One embodiment of the present invention provides a method of screening for a therapeutic composition useful for treating IBD by: (a) administering a therapeutic composition to an animal that serves as a model of IBD and that has strain 1687A6 in its GI and (b) assaying the effect of the therapeutic composition on the levels of strain 1687A6 in the GI of the animal.

One embodiment of the present invention provides a method of screening for a therapeutic composition useful for treating IBD by: (a) administering a therapeutic composition to an animal that serves as a model of IBD and that has strain 1687A6 in its GI and (b) assaying the effect of the therapeutic composition on the levels of Th17 cells in the GI of the animal.

One embodiment of the present invention provides a method of screening for a therapeutic composition useful for treating IBD by: (a) administering a therapeutic composition to a patient diagnosed with IBD and (b) assaying the effect of the therapeutic composition on the levels of strain 1687A6 in the gut of the patient; (c) comparing the levels of strain 1687A6 in the treated patient with levels of strain 1687A6 in a healthy individual; and (d) determining whether the therapeutic composition caused levels of strain 1687A6 to reach a predetermined level in the patient.

One embodiment of the present invention provides a method of screening for a therapeutic composition useful for treating IBD by: (a) administering a therapeutic composition to a patient diagnosed with IBD and (b) assaying the effect of the therapeutic composition on the levels of Th17 cells in the gut of the patient; (c) comparing the levels of Th17 cells in the treated patient with levels of Th17 cells in a healthy individual; and (d) determining whether the therapeutic agent caused levels of Th17 to reach a predetermined level in the patient.

The present invention includes strain 1687A6 or a physiologically active substance derived from strain 1687A6 that causes the proliferation or accumulation of Th7 cells. Immunity in a living organism can be suppressed or increased through administration of therapeutic compositions that inhibit strain 1687A6 or the physiologically active substances derived from it. These therapeutic compositions can be provided as a pharmaceutical product, probiotic, food, beverage, or other biologically active. Accordingly, the composition of the present invention can be used, for example, to identify compositions for the diagnosis, prevention or treatment of IBD. As a result, it is possible to reduce or inhibit the proliferation or accumulation of Th17 cells in the GI and thereby to improve patient prognosis and outcome using the present invention.

Similarly, the immune response in a living organism can be increased through administration of therapeutic compositions that contain strain 1687A6 or physiologically active substances derived from strain 1687A6. These therapeutic compositions can be provided as a pharmaceutical product, biological, probiotic, food, beverage, fecal transfer, or other biologically active substance. Accordingly, the composition of the present invention can be used, for example, to stimulate Th17 cell proliferation or accumulation in an organism. It is therefore possible, for example, to administer such a therapeutic composition to treat diseases, enhance the effectiveness of agents used to treat a specific disease, or identify compositions that modulate immune responses in a subject.

One embodiment of the present invention provides a method for modulating an immune response in a subject by administering a therapeutic composition comprising: (a) the bacterial strain 1687A6; (b) at least one physiologically active substance derived from strain 1687A6; or (c) a complex microbial or fecal sample containing strain 1687A6.

One embodiment of the present invention provides a method for inducing proliferation or accumulation of Th17 cells in a subject by administering a therapeutic composition comprising: (a) the bacterial strain 1687A6; (b) at least one physiologically active substance derived from strain 1687A6; or (c) a complex microbial or fecal sample containing strain 1687A6.

One embodiment of the present invention provides a method for identifying a physiologically active substance derived from strain 1687A6 that induces the differentiation, accumulation, or proliferation of Th17 cells in a subject comprising: (a) generating a strain 1687A6 culture; (b) obtaining from the culture bacterial and supernatant extracts, which contain substances present within, secreted by, produced by, or situated on the surface of strain 1687A6 cells; (c) testing whether a particular extract or combination of extracts induces the accumulation of or proliferation of Th17 cells in a subject.

One embodiment of the present invention provides a method for identifying a physiologically active substance derived from strain 1687A6 that induces Th17 cell proliferation, accumulation, or differentiation comprising: (a) generating a strain 1687A6 culture; (b) obtaining from the culture bacterial and supernatant extracts, which contain substances present within, secreted by, produced by, or situated on the surface of strain 1687A6 cells; (c) contacting a T-Cell population with an extract and determine whether the extract induces the differentiation, accumulation, or proliferation of Th17 cells in the T cell population.

One embodiment of the present invention provides a method for identifying a physiologically active substance derived from strain 1687A6 that induces Th17 cell proliferation, accumulation, or differentiation comprising: (a) generating a strain 1687A6 culture; (b) obtaining from the culture bacterial and supernatant extracts, which contain substances present within, secreted by, produced by, or situated on the surface of strain 1687A6 cells; (c) contacting a T-Cell population with an extract and determine whether the extract induces the activation of Th17 cell associated genes.

One embodiment of the present invention provides a method for identifying a physiologically active substance derived from strain 1687A6 that induces Th17 cell proliferation, accumulation, or differentiation in a subject comprising: (a) sequencing the genome of strain 1687A6; (b) comparing the genome of strain 1687A6 with the genome of at least one E. coli strain that does not normally induce Th17 cell proliferation, accumulation, or differentiation; (c) identifying the differences in nucleotide sequences between the two strains; (d) use recombinant technology to express strain 1687A6 specific sequences in an E. coli strain that does not normally induce Th17 cell proliferation, accumulation, or differentiation; and (e) testing whether the recombinant E. coli induces Th17 cell proliferation, accumulation, or differentiation in a subject.

One embodiment of the present invention provides a therapeutic composition containing strain 1687A6 or at least one physiologically active substance derived from strain 1687A6, wherein the composition comprises a: vaccine; adjuvant; biological; pharmaceutical composition, probiotic; food; beverage; fecal transplant; or a reagent used in an animal model, or a combination of such ingredients.

One embodiment of the present invention provides a method for enhancing the effectiveness of a therapeutic composition comprising: (a) administering to a patient an active therapeutic agent used for treating a specific disease; and (b) co-administering a therapeutic composition consisting essentially of strain 1687A6 or at least one physiologically active substance derived from strain 1687A6, wherein the co-administration occurs, before, after, or at the same time as the administration of the active therapeutic agent.

Such active therapeutic agents can include, for example, pharmaceutical agents, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal antibodies, vaccines, antibiotics, anti-cancer agents, and combinations thereof.

### BRIEF DESCRIPTION OF THE FIGURES

FIG. 1 shows the induction of Intestinal Th17 cells in germ-free mice by human fecal microbiotas. Germ free C57B1/6 mice were colonized with human fecal microbiota from 18 different donors. After 5-8 weeks, the proportion of IL-17A⁺ (A,B) and RORγt⁺ (C,D) CD4⁺ T cells in the colon (A,C) and ileum (B,D) lamina propria was measured by flow cytometry. IL-17A⁺ T cells were measured following ex vivo restimulation for 4 hours with PMA and ionomycin in the presence of monensin. Each point represents one mouse and black bars show the arithmetic mean +/− the standard error of the mean for each group of mice. The color of the symbols represents the disease status of the donor (healthy, ulcerative colitis, or Crohn's disease) and the symbol shape represents if the microbiota tested was a total fecal microbiota or a cultured collection of microbes from that donor.

FIG. 2 Combinatorial gnotobiotics identifies a strain of E. coli that induces colonic Th17 cells and worsens experimental colitis. The figure shows the results of high throughput anaerobic culturing combined with gnotobiotic mouse screening to identify a strain of E. coli that induces colonic Th17 cells and worsens experimental colitis. In pane 2A, the cultured fraction of one donor's microbiota (donor 87 ) recapitulates the Th17 phenotype observed with the total fecal microbiota.

FIG. 2B Combinatorial gnotobiotics identifies a strain of E. coli that induces colonic Th17 cells and worsens experimental colitis (in pane 2F-H). The figure shows the capacity of 16 isolates from a single donor with Crohn's disease to induce Th17 cells. The 16 isolates from donor 87 were recombined into 8 sub-communities and the capacity of each to induce Th17 cells was tested. The table shows which isolates were included in each sub-community (+). The graph on the right of the table shows the proportion of IL-17A⁺ CD4⁺ T cells in mice colonized with each sub-community. The graph under the table shows the p-value of the association between each microbe and the colon Th17 cells. Data is pooled from two independent experiments.

FIGS. 2C, 2D show E. coli_A6 is strongly and uniquely associated with colonic IL-17A+ and RORγt+ T cells. Data in C and D is pooled from two independent experiments and each point represents data from one mouse. Data in C and D are not normally distributed. P values are calculated using Mann-Whitney test. FIG. 2C shows the association of a specific isolate (E. coli_A6 ) with colonic IL-17A⁺ cells. FIG. 2D shows the association of a specific isolate (E. coli_A6 ) with colonic RORγt⁺ T cells.

FIG. 2E Combinatorial gnotobiotics identifies a strain of E. coli that induces colonic Th17 cells and worsens experimental colitis. The figure shows that a community of bacteria isolated from a single donor (donor 87 ) but that lack E. coli A6 fails to induce colonic Th17 cells. It also shows that another strain of E. coli isolated from donor 87 (E. coli E2) is not associated with colonic Th17 cells. A community of all donor 87 isolates but lacking E. coli_A6 fails to induce colonic Th17 cells. Another strain of E. coli isolated from donor 87 (E. coli_E2) is not associated with Th17 cells. Each point represents one mouse and is data pooled from two independent experiments. * p<0.01.

FIG. 2F, G, H show that mice colonized with E. coli_A6 exhibit more severe intestinal inflammation induced by 2.5% DSS in drinking water. Combinatorial gnotobiotics identifies a strain of E. coli that induces colonic Th17 cells and worsens experimental colitis. FIG. 2F shows that mice colonized with E. coli_A6 exhibit more severe intestinal inflammation induced by 2.5% DSS in drinking water—by mean change in body mass +/− SEM. Representative of two independent experiments with similar results. * p=0.04, ** p=0.0014 at day 7, as calculated by ANOVA with Tukey's correction for multiple comparisons. FIG. 2G shows that mice colonized with E. coli_A6 exhibit more severe intestinal inflammation induced by 2.5% DSS in drinking water—he colon length of individual mice. Pooled from two independent experiments, each point represents the length of the colon of one mouse at day 7 after start of DSS treatment. ** p=0.0014, *** p=0.0007 calculated by ANOVA with Tukey's correction for multiple comparisons. FIG. 2H shows that mice colonized with E. coli_A6 exhibit more severe intestinal inflammation induced by 2.5% DSS in drinking water—by colon histopathology score. Scored by a pathologist blinded to the experimental conditions * p=0.04.

### BRIEF DESCRIPTION OF THE SEQUENCES

Escherichia coli strain 1687A6 is defined by a 4.9 megabase genome. This genome sequence can be used to track the strain using metagenomics sequencing or cultured isolate sequencing.

### DETAILED DESCRIPTION

Note that the term “individual” in the present invention is not particularly limited, and examples thereof include humans, mice, rats, cattle, horses, pigs, sheep, monkeys, dogs, and cats.

The term “therapeutic composition” according to the present invention may be in the form of a vaccine, adjuvant, biological, pharmaceutical composition, probiotic, food, beverage, fecal transplant, complex microbial or fecal sample, a reagent used in an animal model, or a combination of such ingredients. The vaccine, adjuvant, biological, pharmaceutical composition, probiotic, food, beverage, or reagent, or combinatorial product can have the effect of reducing or eliminating strain 1687A6 or of physiologically active substances derived from strain 1687A6 in the GI of a subject. The therapeutic compositions according to the present invention can also have the effect of stimulating or enhancing the differentiation, accumulation, or proliferation of Th17 cells in a subject or of stimulating the immune response in a subject. Administration of such therapeutic compositions may be oral, buccal, parenteral, rectal, or via fecal transplantation.

The term “IBD” in the present invention includes gastrointestinal disorders such as inflammatory bowel disease (IBD), ulcerative colitis, and Crohn's disease.

The term “Th17” or “Th17 cells” means CD4⁺ TH17 cells that express the transcription factor RORγt⁺ T, produce the cytokine IL-17, and play a critical role in promoting homeostasis at the mucosal barrier in the gut.

The phrase “reducing or eliminating differentiation, accumulation, or proliferation of Th17 cells” in the present invention includes an effect of reducing or inhibiting the differentiation of immature T cells into Th17 cells, which differentiation leads to the proliferation or the accumulation of Th17 cells. In addition, the meaning of the “reducing or eliminating differentiation, accumulation, or proliferation of Th17 cells” in the present invention includes in-vivo effects, in vitro effects, and ex vivo effects. Accordingly, all of the following effects are included: reducing or inhibiting in vivo proliferation or accumulation of Th17 cells in the gut through administration or ingestion of a therapeutic composition that inhibits strain 1687A6 or inhibits a physiologically active substance derived from strain 1687A6; reducing or inhibiting proliferation or accumulation of Th17 cells by preventing strain 1687A6 or a physiologically active substance derived from the bacteria to act on the cultured Th17 cells; and reducing or inhibiting proliferation or accumulation of Th17 cells that are collected from a living organism and that are intended to be subsequently reintroduced into that organism or introduced into another organism, by preventing strain 1687A6 or a physiologically active substance derived from the bacteria to act on the Th17 cells.

The effect of reducing or inhibiting differentiation, proliferation, or accumulation of Th17 cells can be evaluated, for example, by: orally administering strain 1687A6 to an experimental animal such as a germ-free mouse, allowing strain 1687A6 to proliferate in the GI of the animal, administering a therapeutic composition to the animal, isolating CD4-positive cells from the GI, measuring by flow cytometry the ratio of Th17 cells contained in the CD4-positive cells, and comparing the post-administration Th17 ratio to the pre-administration ratio or a predetermined level of Th17 cells.

One can determine whether the “reducing or eliminating differentiation, accumulation, or proliferation of Th17 cells” is occurring, for example, by assaying the ratio of Th17 cells in the T cell group of the colon, a function of Th17 cells in the colon, or expression of a marker of Th17 cells in the colon, such as RORγt⁺.

Methods of detecting Th17 cell RNA expression markers include, for example, high throughput RNA screening, northern blotting, dot blotting, and RT-PCR. Examples of methods for detecting protein markers include, for example, ELISA, radioimmunoassay, immunoblotting, immunoprecipitation, and flow cytometry.

Methods of screening for strain 1687A6 specific nucleotides include, for example, high throughput DNA or RNA screening, Southern blotting, northern blotting, dot blotting, recombinant DNA expression, and RT-PCR. Examples of methods for detecting strain 1687A6 specific protein markers include, for example, ELISA, radioimmunoassay, immunoblotting, and immunoprecipitation.

The meaning of “physiologically active substance derived from strain 1687A6 and “physiologically active substance derived from the bacteria” of the present invention includes metabolites of the bacteria and substances: contained in the bacteria; secreted by the bacteria; or affixed to the surface of the bacteria. Such a physiologically active substance can be identified by purifying an active component from the bacteria or supernatants of bacterial cultures or from the intestinal tract of a mouse colonized only by strain 1687A6. It can also be identified by screening for strain 1687A6 specific nucleotide sequences that lead to the differentiation, proliferation, or accumulation of Th17 cells in a subject.

The present invention can provide methods for determining the effects of a therapeutic composition by measuring the absolute amount or the ratio of strain 1687A6 in a microbiota of an individual diagnosed with IBD, treating the individual with a therapeutic composition, and evaluating whether the absolute amount or ratio of strain 1687A6 is reduced in comparison with a base line value obtained by performing a similar evaluation on a healthy individual.

One embodiment of the present invention provides a method for predicting a patient's response to a therapeutic composition and provide a prognosis. The method comprises measuring the percentage or absolute amounts strain 1687A6 in the microbiota in a subject diagnosed with IBD. Comparing it to a baseline value for those amounts in a healthy subject. Combining the results of the comparison with additional diagnostic information and medical history data related to the patient. Evaluating whether the patient may show a reduction in IBD after administration of a particular therapeutic composition.

### EXAMPLES

Provided below are select examples of certain embodiments of the present invention; however, the invention is not limited to these examples or the specific embodiments recited above.

The inventors have identified a single bacterial strain that is a component of human gut microbiotas and that regulates different aspects of intestinal T cell function. To identify this bacterial strain, the inventors colonized germ free mice (which are born and raised under sterile conditions and have no pre-existing microbiota) with human gut microbiotas. The inventors used flow cytometry to study how these microbiotas changed the tone of the T cell response in the gut tissue of the mice.

Stool samples from inflammatory bowel disease patients and healthy controls were processed under anaerobic conditions. Using a wide range of solid media and culture conditions, a diverse selection of microbes were isolated from each sample and cultured in multiwell formats. Each isolate was identified using a combination of MALDI-TOF mass spectrometry, 16S rRNA and whole-genome sequencing. Microbes were pooled or selectively recombined and introduced to germ-free mice to generate personalized, humanized gnotobiotic mice.

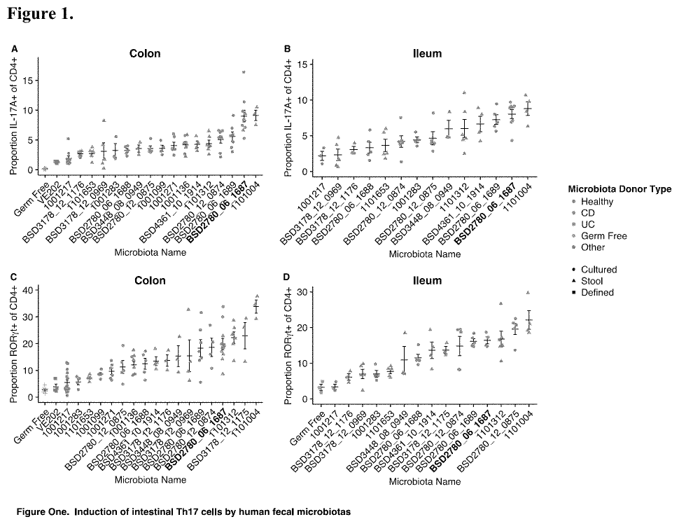

Microbiotas were obtained from healthy donors and donors with ulcerative colitis (UC) or Crohn's disease (CD). Eighteen (18) different complex fecal microbiotas from each human donor were screened. Of these, microbiotas from two donors, both individuals with Crohn's disease, induced a greater proportion of IL-17A-secreting Th17 cells in the colon and small intestine of mice than all other donors tested. (See FIG. 1.)

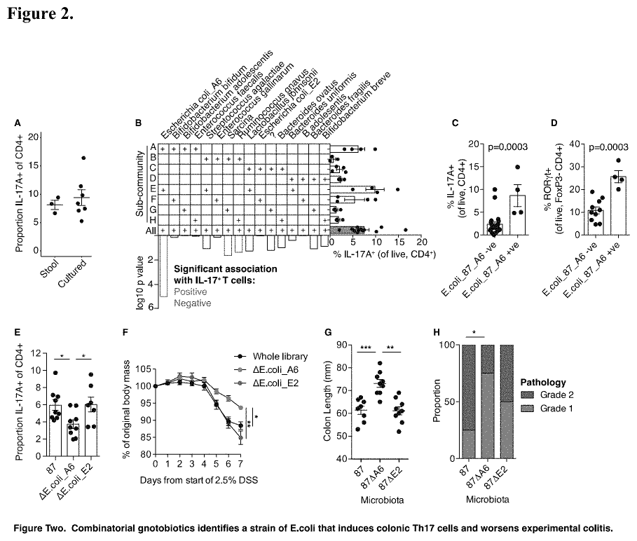

The inventors subjected the complex fecal microbiota of one donor with Crohn's disease (“donor 87”) to high throughput anaerobic culturing and generated a diverse cultured collection of bacteria from this donor. Using 16s rDNA and whole genome sequencing, the inventors identified 16 unique isolates from the microbiota of donor 87 . The inventors subsequently colonized germ-free mice with a pool of the isolates and surprisingly discovered that this culturable fraction of the microbiota from donor 87 recapitulated the elevated Th17 cell response observed with the complex fecal microbiota from the donors. (See FIG. 2A.) At the time of sampling, the Crohn's disease in donor 87 was in remission.

The inventors recombined the isolates into 8 new sub-communities using an orthogonal screen design. Each sub-community comprised four microbes, with each microbe appearing in two subcommunities. The inventors subsequently colonized groups of germ-free mice with each of the 8 sub-communities and assessed Th17 cell induction in each group. Th17 cells were induced by two of the subcommunities. One strain of Escherichia coli (“E. coli_A6”) featured in both communities and in no other. Linear modeling confirmed that E. coli_A6 was the only isolate that had a significant positive correlation with the proportion of colonic Th17 cells induced. (See FIGS. 2B, 2C, and 2D.)

The inventors discovered that E. coli_A6 was necessary for Th17 induction by colonizing mice with either the entire cultured collection of microbes, the collection of microbes without E. coli_A6, or the collection of microbes lacking a different strain of E. coli isolated from donor 87 (referred to as E. coli E2). E. coli E2 showed no significant correlation with Th17 cells in the combinatorial screen. In agreement with the screening data, the collection of microbes lacking E. coli_A6 induced a lower proportion of colonic Th17 cells than either the entire cultured collection or the microbe collection lacking E. coli_E2. (See FIG. 2E.)

The inventors further discovered that the increased proportion of Th17 cells induced by donor 87, and specifically by E. coli_A6, renders mice more susceptible to intestinal inflammation. The inventors colonized germ free mice with either (a) the cultured collection of microbes from donor 87 or (b) the collections lacking either strain of E. coli. The inventors induced intestinal inflammation using dextran sodium sulfate, administered in drinking water. Colitis was less severe in mice lacking E. coli_A6, as measured by reduced weight loss, increased colon length, and less severe histological pathology. (See FIGS. 2F, 2G, and 2H).

1. What is claimed is: A method of detecting strain 1687A6 in a patient comprising:
obtaining a fecal sample from a human patient and screening for the presence of strain 1687A6 specific polynucleotides or polypeptides in the sample.

2. The method of claim 1 further comprising isolating nucleic acid sequences present in the fecal sample; and screening for the presence of strain 1687A6 specific sequences in the sample.

3. The method of claim 1 further comprising contacting the sample with an anti-strain 1687A6 antibody; and detecting whether strain 1687A6 is present in the sample by detecting binding between strain 1687A6 and the antibody.

4. The method of claim 1 further comprising detecting strain 1687A6 specific RNA sequences in the sample.

5. A method of determining the levels of strain 1687A6 in the gastrointestinal tract of a patient with inflammatory bowel disease comprising: obtaining a fecal sample from a human patient; isolating RNA from the sample; detecting strain 1687A6 specific RNA in the sample; and comparing the amount of strain 1687A6 specific RNA in the sample to a predetermined level of strain 1687A6 specific RNA.

6. A method of diagnosing IBD in a patient comprising: obtaining a fecal sample from a human patient; isolating nucleotides from the sample; detecting strain 1687A6 specific nucleotides in the sample; and comparing the amount of strain 1687A6 specific nucleotides in the sample to a predetermined level of strain 1687A6 specific nucleotides.

7. A method of identifying a therapeutic composition for treating IBD comprising: (a) determining the amount of strain 1687A6 in the GI of an individual; (b) administering a therapeutic composition to the individual; (c) assaying the effect of the therapeutic composition on the levels of strain 1687A6 or of Th17 cells in the GI of the individual;
(c) comparing the levels of strain 1687A6 in the treated individual with predetermined levels of strain 1687A6 or of Th17 cells in the treated individual with predetermined levels of Th17 cells; and (d) determining whether the therapeutic agent reduces the levels of strain 1687A6 or of Th17 cells in the individual below the predetermined level.

8. The method of claim 7 wherein the method is limited to detecting and determining the levels of Th17.

9. The method of claim 7 wherein the method is limited to detecting and determining the levels of strain 1687A6.

10. A method for modulating an immune response in a subject by administering to a subject a therapeutic composition comprising: (a) the bacterial strain 1687A6; (b) at least one physiologically active substance derived from strain 1687A6; or (c) a fecal sample containing strain 1687A6.

11. The method of claim 10 wherein the immune response comprises inducing differentiation, proliferation, or accumulation of Th17 cells in the subject.

12. A method for identifying a physiologically active substance derived from strain 1687A6 that induces Th17 cell differentiation, proliferation, or accumulation in a subject comprising: (a) sequencing the genome of strain 1687A6; (b) comparing the genome of strain 1687A6 with the genome of at least one E. coli strain that does not normally induce Th17 cell proliferation, accumulation, or differentiation; (c) identifying the differences in nucleotide sequences between the two strains; (d) use recombinant technology to express strain 1687A6 specific sequences in an E. coli strain that does not normally induce Th17 cell proliferation, accumulation, or differentiation; and (e) testing whether the recombinant E. coli induces Th17 cell proliferation, accumulation, or differentiation in a subject.

13. A therapeutic composition containing strain 1687A6 or at least one physiologically active substance derived from strain 1687A6, wherein the composition comprises a: vaccine;
adjuvant; biological; pharmaceutical composition, probiotic; food; beverage; fecal transplant; or a reagent used in an animal model, or a combination of such ingredients.

14. A method for treating a disease in a subject by administering a therapeutic composition containing strain 1687A6 or at least one physiologically active substance derived from strain 1687A6, wherein the composition comprises a: vaccine; adjuvant; biological;
pharmaceutical composition, probiotic; food; beverage; fecal transplant; a reagent used in an animal model; or a combination of such ingredients.

15. The method of claim 14 wherein the disease is an infectious disease.
